(19) United States
(12) Patent Application Publication
(10) Pub. No.: US 2008/0260771 A1
(43) Pub. Date: Oct. 23, 2008

(54) PROSTATE DISORDER(S) PHYTO-NUTRACEUTICAL SYNERGISTIC COMPOSITION

(76) Inventor: JOSE ANGEL OLALDE RANGEL, Clearwater, FL (US)

Correspondence Address: JOSE OLALDE RANGEL, 519 CLEVELAND STREET, SUITE 101, CLEARWATER, FL, 33755, US

(21) Appl. No.: 11/530,934

(22) Filed: Sep. 12, 2006

(51) Int. Cl. A61K 36/06; A61K 33/04; A61K 31/355; A61K 33/32; A61K 36/254; A61K 36/42; A61K 36/28; A61K 36/87

(52) U.S. Cl. 424/195.15; 424/728; 424/702; 424/766; 424/758; 424/641; 514/458; 424/764

(57) ABSTRACT

A Phyto-nutraceutical composition for the prevention and treatment of prostate disorders is provided. A specific combination of extracts of plants and nutraceuticals is provided, based on categorizing plants and nutraceuticals into one of three groups, Energy, Bio-Intelligence, and Organization. Such combinations have synergistic effects, with minimal side effect.

### PRIOR RELATED APPLICATIONS

Not applicable.

### FEDERALLY SPONSORED RESEARCH STATEMENT

Not applicable.

### REFERENCE TO MICROFICHE APPENDIX

Not applicable.

### FIELD OF THE INVENTION

The invention relates to a phytoceutical formulation used to treat prostate disorders. The formulation is a particular combination of plants that have synergistic effect in combination. Principles for selecting beneficial formulations are provided.

### BACKGROUND OF THE INVENTION

The academic study of medicinal plants for the treatment of diverse diseases has been nearly as pervasive as the study of Western medicines. The active principles from many traditional medicines have been extracted from plants, the curative agents identified and their mechanisms of action determined. Plant based medicines are typically well tolerated, with less severe side effects as well as a smaller range of side effects. In contrast, while synthetic drugs can be highly effective, their use is often hampered by severe side effects. Additionally, while synthetic pharmaceuticals are based upon single chemicals, many phytomedicines exert their beneficial effects through the additive or synergistic action of several chemical compounds acting at single or multiple target sites associated with a physiological process.

As pointed out by Tyler (1999), this synergistic or additive pharmacological effect can be beneficial by eliminating the problematic side effects associated with the predominance of a single xenobiotic compound in the body. In this respect, Kaufman et al. (1999) extensively documented how synergistic interactions underlie the effectiveness of a number of phytomedicines. A more recent study, with additional demonstration of a phytomedicine's synergistic effect—Echinacea—is provided by Dalby-Brown et al, 2005. This theme of multiple chemicals acting in an additive or synergistic manner likely has its origin in the functional role of secondary products in promoting plant survival. For example, in the role of secondary products as defense chemicals, a mixture of chemicals having additive or synergistic effects at multiple target sites would not only ensure effectiveness against a wide range of herbivores or pathogens but would also decrease the chances of these organisms developing resistance or adaptive responses (Kaufman et al., 1999; Wink, 1999). Conclusion: On one hand, synthetics may have the required efficacy for disease treatment; however this can be marred by severe side effects. On the other hand, despite the excellent medicinal qualities of many plants, they are individually insufficient to take chronic degenerative diseases into remission. However, there is mounting evidence which demonstrates that medical plants contain synergistic efficacy and/or side-effect neutralizing combinations (Gilani and Rahman, 2005). Thus, what are needed in the art are better treatment regimes with improved patient tolerance, while providing sufficient efficacy.

### SUMMARY OF THE INVENTION

A number of known beneficial plants and tonics were classified according to their capacity to enhance the three main elements that support overall health: Energy (E), Bio-intelligence (I) and Organization (O). A synergistic effect is expected when all three categories of herbs and tonics (E, I, O) are included in a formulation, preferably at least two or three or four plants from each category. Thus, the invention provides the selection of disease treating formulations according to these principles. An example of a formulation prepared this way is provided and additional formulations are being prepared and tested.

Another embodiment of the invention provides an effective, natural composition for treating prostate disorder(s) and symptoms. The composition can be used alone, or can be combined simultaneously with one or more pharmaceutical compositions.

### DETAILED DESCRIPTION OF THE INVENTION

“Pharmaceutically acceptable excipients” is used herein according to art accepted meanings, and includes those ingredients needed to formulate a medicine for mammalian use, including the use of gelatin capsules.

“Synergistic” or “synergy” is used herein to mean that the effect is more than its additive property. In preferred embodiments, the synergy is at least 1.5, 2, 5, or 10 fold.

By use of “plants,” what is meant herein is that the plant (or that portion with medicinal activity) is used whole, ground, or as an extract. Also included are purified active ingredients and derivatives thereof. However, it is believed that the best efficacy of plants used herein is achieved with the use of the entire plant or its extracts, rather than with the use of isolated active ingredients.

Further, although plants are named here according to commonly used nomenclature, with improving taxonomy plants are often reclassified. Whenever a plant is referenced, it includes related species with similar active ingredients.

The following examples are illustrative only and should not serve to unduly limit the invention.

### EXAMPLE 1

### Plant Characteristics—Prostate Disorders

Energy Enhancing Components.—

Panax ginseng (Chinese ginseng, panax, ren shen, jintsam, ninjin, Asiatic ginseng, Japanese ginseng, Oriental ginseng, Korean red ginseng) The main active components are ginsenosides (protopanaxadiols and protopanaxatriols types) these have been shown to have a variety of beneficial effects, including anti-inflammatory, antioxidant, and anticancer effects. They also confer energizing properties because they increase ATP synthesis. Results of clinical research studies demonstrate that Panax may improve immune function. For example, ginsenoside RH2 induces apoptosis via activation of caspase-1 and caspase-3 and upregulation of Bax in human neuroblastoma. Also, diets containing ginseng, decreased/modulated the numbers of aberrant fossi. Thus, this herbal supplement may exert significant and potentially beneficial effects on decreasing the amount of precancerous lesions and inducing apoptosis. Studies indicate that Panax enhances phagocytosis, NK lymphocytes cell activity, and the production of interferon; improves physical and mental performance in mice and rats; and increases resistance to exogenous stress factors. The incorporation of this phytomedicine provides at least 86 active principles in a single therapeutic.

Panax quinquefolius (American Ginseng, Anchi, Canadian Ginseng, Five Fingers, Ginseng, American, North American Ginseng, Red Berry, Ren Shen, and Tienchi) is related to Panax ginseng, but is a distinct species with higher levels of ginsenoside Rb1 and without ginsenoside Rf. These substances confer energizing properties because they increase ATP synthesis. Ginsenoside Rb1 is believed to limit or prevent the growth of new blood vessels, making it useful to treat tumors. It has antioxidant, anti-inflammatory, and hypolipidemic effects. Studies revealed that quinquefolius and estradiol equivalently induced RNA expression of pS2. Panax, in contrast to estradiol, caused a dose-dependent decrease in cell proliferation. Quinquefolius had no adverse effect on the cell cycle while estradiol significantly increased the proliferative phase (percent S-phase) and decreased the resting phase—G(0)-G(1) phase. Concurrent use of quinquefolius and breast cancer therapeutic agents resulted in a significant suppression of tumoral cell growth for most drugs evaluated. This phytomedicine provides at least 206 active principles in a single therapeutic.

Rhaponticum carthamoides (Leuzea carthamoides, or Maral Root) contains a mixture of compounds called ‘levseins’. Levseins represents a complex of more than 10 ecdysterones including 20-beta-ecdysterone, makisterone C, 24-dehydromakisterone A, carthamosterone, polypodyne B and ajugasterone C. Researchers extracted and purified various ecdysteroids from Rhaponticum and found that the ecdysteroids increased the muscle mass in a dose-dependent manner, with the rate of increase proportional to the ecdysteroids content. Ecdysteroids normalize NADH dehydrogenase activity, enzyme which catalyses NADH electron transfer to the ubiquinone in the oxidative phosphorylation processes at the mitochondrial level, contributing to buildup the electrochemical potential used to produce ATP. It also normalizes the succinate dehydrogenase activity, enzyme which acts in the tricarboxilic acid cycle, which translates in ATP synthesis and patient energy level increases [Tashmukhamedova M A, Almatov K T, Syrov V N. Comparative study of the effect of ecdysterone, turkesterone and nerobol on the function of rat liver mitochondria in experimental diabetes. Vopr Med Khim. 1986; 32:24-8]. Incorporation of this phytomedicine provides at least 10 active principles in a single therapeutic.

Bio-Intelligence Modulators.—

Ganoderma lucidum (Reishi, also G. tsugae, G. valesiacum, G. oregonense, G. resinaceum, G. pfezfferi, G. oerstedli, and G. ahmadii) is an edible fungus containing bitter triterpenoids (ganoderic acid), β-D-glucan, coumarins, alkaloids and ergosterols. The main active principles of this mushroom are sterols and beta-proteoglucans which bestow anti-inflammatory and immune-modulating properties, because they increase the phagocytotic capacity of macrophages, and increase the production—and lifetime—of CD4 lymphocytes as well. Treatment with beta-glucan may be beneficial for cancer patients with or at risk for metastasis. The beta-glucan-dependent signaling pathways are critical for our understanding of anticancer events and development of cancer therapeutic agents. The polysaccharide component with a branched (1-->6)-beta-D-glucan moiety of G. lucidum (PS-G) has been reported to exert anti-tumor activity and activation of natural killer cells. Also data suggests that can effectively promote the activation and maturation of immature dendritic cells suggesting that ganoderma may posses a potential in regulating immune responses. The extract of Ganoderma lucidum showed strong 5alpha-reductase inhibitory activity. Significantly inhibited the testosterone-induced growth of the ventral prostate in castrated rats.

These results showed that Ganoderma lucidum might be a useful ingredient for the treatment of benign prostatic hyperplasia (BPH) (Fujita R, Liu J, Shimizu K. Anti-androgenic activities of Ganoderma lucidum. J Ethnopharmacol. 2005; 102:107-12). G. lucidum induces apoptosis, inhibits cell proliferation, and suppresses cell migration of highly invasive human prostate cancer cells. It also inhibits the early event in prostate cancer-dependent angiogenesis, capillary morphogenesis of the human endothelial cells (Stanley G, Harvey K, Slivova V. Ganoderma lucidum suppresses angiogenesis through the inhibition of secretion of VEGF and TGF-beta1 from prostate cancer cells. Biochem Biophys Res Commun. 2005; 330:46-52). Ganoderma lucidum down-regulates the expression of NF-kappaB-regulated urokinase plasminogen activator (uPA) and uPA receptor (uPAR), which results in suppression of cell migration of highly invasive prostate cancer cells. Ganoderma lucidum inhibits cell proliferation in a dose- and time-dependent manner by the down-regulation of expression of cyclin B and Cdc2 and by the up-regulation of p21 expression. The inhibition of cell growth was also demonstrated by cell cycle arrest at G2/M phase. Furthermore, Ganoderma lucidum induced apoptosis of prostatic cancer cells with a slight decrease in the expression of NF-kappaB-regulated Bcl-2 and Bcl-xl. However, the expression of proapoptotic Bax protein was markedly up-regulated, resulting in the enhancement of the ratio of Bax/Bcl-2 and Bax/Bcl-xl (Jiang J, Slivova V, Valachovicova T. Ganoderma lucidum inhibits proliferation and induces apoptosis in human prostate cancer cells PC-3. Int J Oncol. 2004; 24:1093-9). Ganoderma contains at least 32 active principles.

Grifola frondosa (Maitake, Dancing Mushroom; also G. sordulenta, Polyporus umbellatus and Meripilus giganteus) contains the primary polysaccharide, β-D-glucan in the 1.3 and 1.6 forms. It also contains alpha glucan, lipids, phospholipids, and ergosterol. β-D-glucan is recognized as an effective immuno-stimulator. This substance increases the activity of macrophages and other immunocompetent cells that destroy tumor cells. The substance also improves the immunological efficiency of these cells by increasing production of cytokines IL-1, IL-2 and others. The final result is an increase of the defenses against infectious and tumoral diseases.

Also, D-Fraction, a polysaccharide extracted from maitake mushrooms (Grifola frondosa), has been reported to exhibit an antitumoral effect through activation of immunocompetent cells, including macrophages and T cells, with modulation of the balance between T-helper 1 and 2 cells. Study results suggest that D-Fraction can decrease the effective dosage in tumor-bearing mice by increasing the proliferation, differentiation, and activation of immunocompetent cells and thus provide a potential clinical benefit for patients with cancer. A bioactive beta-glucan from the Maitake mushroom has a cytotoxic effect, presumably through oxidative stress, on prostatic cancer cells in vitro, leading to apoptosis. Therefore, this unique mushroom polysaccharide may have great a potential as an alternative therapeutic modality for prostate cancer (Fullerton S A, Samadi A A, Tortorelis D G. Induction of apoptosis in human prostatic cancer cells with beta-glucan (Maitake mushroom polysaccharide). Mol Urol. 2000; 4:7-13). The incorporation of this phytomedicine provides at least 6 active ingredients for therapeutic use.

Vitex agnus castus: From the fruit of this plant are obtained: two iridoid glicosides (aucubine and agnuside); essential oils, a flavone (casticine, which seems to be the main active principle) and three minor flavonoids derived from kaempferol and quercetin. Prolactine in a hormone which increases testosterone binding to the prostate epithelium and stimulates the conversion of testosterone to dihydrotestosterone. Prostatic Nodular Hyperplasia appears to be stimulated by high levels of prolactin. Prolactin-prostate carcinoma interaction involve considering this hormone as a possible carcinogenic agent, emphasizing the existence of high plasma levels of this hormone in individuals with prostate carcinoma. Also, any androgen excess is considered a major factor at the genesis of this tumor, and prolactin increases androgens testicular synthesis. For these reasons, controlling this hormone levels becomes an element to have into consideration in the treatment and follow-up of patients with nodular hyperplasia or carcinoma of the prostate (Romero L, Munoz C, Lopez A. Relation of prolactin with nodular hyperplasia and carcinoma of the prostate. Actas Urol Esp. 1991; 15:503-9).

Another clinical study, placebo-controlled in humans, showed that Vitex agnus castus contains active principles that bind to the pituitary Dopaminergic D2 receptors, inhibiting significantly Prolactin release (Merz P G, Gorkow C, Schrodter A. The effects of an Agnus castus extract on prolactin secretion in healthy male subjects. Exp Clin Endocrinol Diabetes. 1996; 104:447-53).

Organizational Improvers.—

Arcostaphylos uva ursi (bearberry) Contains arbutin, phenolic glucosides and iridoid substances which have demonstrated antiseptic, diuretic and anti-inflammatory action (Kruszewska H, Zareba T, Tyski S. Examination of antimicrobial activity of selected non-antibiotic drugs. Acta Pol Pharm. 2004; 61:18-21) (Yarnell E. Botanical medicines for the urinary tract. World J Urol. 2002; 20:285-93) (Beaux D, Fleurentin J, Mortier F. Effect of extracts of Orthosiphon stamineus Benth, Hieracium pilosella L., Sambucus nigra L. and Arctostaphylos uva-ursi (L.) Spreng. in rats. Phytother Res. 1999; 13:222-5) (Matsuda H, Nakata H, Tanaka T, Kubo M. Pharmacological study on Arctostaphylos uva-ursi (L.) Spreng. II. Combined effects of arbutin and prednisolone or dexamethazone on immuno-inflammation. Yakugaku Zasshi. 1990; 110:68-76).

Cucurbita pepo (Cucurbitaceae—Curcubita pepo L. common name: Pumpkin) The seeds of Pumpkin have a high content of free fatty acids. The content of vitamin E, especially α-tocopherol, is also very high. Its saturated fatty acids are: Palmitic, Myristic, Stearic and Lauric; its unsaturated are: Oleic, Linoleic, Alpha-linolenic, and Palmitoleic. Although the four dominant fatty acids are palmitic, stearic, oleic and linoleic acids, they have all demonstrated capacity to inhibit 5-Alpha-Reductase; enzyme which converts testosterone into dihydrotestosterone, the main hormone associated with prostatic hyperplasia (Weisser H, Tunn S, Behnke B. Effects of the sabal serrulata extract IDS 89 and its subfractions on 5 alpha-reductase activity in human benign prostatic hyperplasia. Prostate. 1996; 28:300-6). Pumpkin also contains phytosterols with anti-inflammatory action, such as alpha-spinasterol (Zhou C C, Sun X B, Liu J Y.

Anti-inflammatory effect of alpha-spinasterol. Yao Xue Xue Bao. 1985; 20:257-61) and beta-sitosterol, which has demonstrated beneficial effects in the treatment of benign prostatic hyperplasia (Berges R R, Kassen A, Senge T. Treatment of symptomatic benign prostatic hyperplasia with beta-sitosterol: an 18-month follow-up. BJU Int. 2000; 85:842-6). It also contains beta-carotene, which modulates the growth of human prostatic cancer cells (Williams A W, Boileau T W, Zhou J R. Beta-carotene modulates human prostate cancer cell growth and may undergo intracellular metabolism to retinol. J Nutr. 2000; 130:728-32). It also contains selenium and zink whose prostatic disease treatment properties have been herein described before. A number of short-term randomized trials and some meta-analyses in the recent literature suggest clinical efficacy for the treatment of Lower Urinary Tract Symptoms and good tolerability for pumpkin seeds (Dreikorn K. The role of phytotherapy in treating lower urinary tract symptoms and benign prostatic hyperplasia. World J Urol. 2002; 19:426-35) Curcubita provides at least 73 active principles in a single therapeutic.

Pygeum africanum (Prunus africanum) Its main active principles are phytosterols, particularly beta-sitosterol, whose beneficial properties in the treatment of Benign Prostatic Hyperplasia have been demonstrated (Berges R R, Kassen A, Senge T. Treatment of symptomatic benign prostatic hyperplasia with beta-sitosterol: an 18-month follow-up. BJU Int. 2000; 85:842-6). It also contains pentacyclic triterpenes, such as ursolic acid which can induce apoptosis in human prostate cancer cells (Choi Y H, Baek J H, Yoo M A. Induction of apoptosis by ursolic acid through activation of caspases and down-regulation of c-IAPs in human prostate epithelial cells. Int J Oncol. 2000; 17:565-71) additionally offering anti-inflammatory properties through inhibition of COX2 (Subbaramaiah K, Michaluart P, Sporn M B. Ursolic acid inhibits cyclooxygenase-2 transcription in human mammary epithelial cells. Cancer Res. 2000; 60:2399-404). In one meta-analysis carried out in the Minneapolis Veterans Affairs Center for Chronic Disease Outcomes Research and the VA Coordinating Center for the Cochrane Review Group in Prostate Diseases and Urologic Malignancies, Minneapolis, USA, a total of 18 randomized controlled trials involving 1,562 men were analyzed.

This study stated that “evidence suggests that P. africanum modestly, but significantly, improves urologic symptoms and flow measures” (Ishani A, MacDonald R, Nelson D, Rutks I. Pygeum africanum for the treatment of patients with benign prostatic hyperplasia: a systematic review and quantitative meta-analysis. Am J Med 2000; 109:654-64). It provides no less than 5 active principles to the formulation.

Selenium is a micronutrient that is incorporated into a number of essential enzymes and a minimum intake is necessary for the maintenance of health. In the last few years evidence has accumulated from clinical, randomized, controlled trials that supranutritional doses of selenium offers significant protective effects on the overall incidence of prostate cancer and inhibits the progression of prostate cancer (Duffield-Lillico A J, Dalkin B L, Reid M E. Selenium supplementation, baseline plasma selenium status and incidence of prostate cancer: an analysis of the complete treatment period of the Nutritional Prevention of Cancer Trial. BJU Int. 2003; 91:608-12). Secondary intervention strategies with selenium compounds and other agents represent a viable option to reduce the morbidity and mortality of prostate cancer (Meuillet E, Stratton S, Prasad Cherukuri D. Chemoprevention of prostate cancer with selenium: an update on current clinical trials and preclinical findings. J Cell Biochem. 2004; 91:443-58). Selenium reduces prostate cancer incidence by 50%. Inhibits human prostate cancer cell growth, blocks cell cycle progression, and induces apoptotic cell death. It also markedly reduces androgen signaling and androgen receptor (AR)-mediated gene expression, including prostate-specific antigen (PSA), in human prostate cancer cells. Selenium disrupts AR signaling at multiple stages, including AR mRNA expression, mRNA stability, protein degradation, nuclear translocation, and recruitment of co-regulators (Chun J Y, Nadiminty N, Lee S O. Mechanisms of selenium down-regulation of androgen receptor signaling in prostate cancer. Mol Cancer Ther. 2006; 5:913-8) (Combs G F Jr. Status of selenium in prostate cancer prevention. Br J Cancer. 2004; 91:195-9). 586 men diagnosed with prostate cancer during 13 years of follow-up and 577 control subjects. Pre-diagnostic plasma selenium levels were inversely associated with risk of advanced prostate cancer.

This suggests that higher levels of selenium may slow prostate cancer tumor progression (Li H, Stampfer M J, Giovannucci E L. A prospective study of plasma selenium levels and prostate cancer risk. J Natl Cancer Inst. 2004; 96:696-703). Another study provides evidence that selenium induces rapid apoptotic death in human prostate cancer cells, but not in normal prostate epithelial cells. Apoptosis involves activation of caspase 3 which plays a critical role in the cell death process (Ghosh J. Rapid induction of apoptosis in prostate cancer cells by selenium: reversal by metabolites of arachidonate 5-lipoxygenase. Biochem Biophys Res Commun. 2004; 315:624-35).

Serenoa repens (Saw palmetto, Sabal serrulata, Scrub-Palmetto) Its main active principles are saturated fatty acids: lauric, myristic, palmitic, capric, caprylic; and unsaturated fatty acids: oleic, linoleic and linolenic. These fatty acids have demonstrated inhibiting action on 5-Alpha-Reductase, enzyme which transforms Testosterone into Dihydrotestosterone-associated with prostatic hiperplacia (Habib F K, Ross M, Ho C K. Serenoa repens (Permixon) inhibits the 5alpha-reductase activity of human prostate cancer cell lines without interfering with PSA expression. Int J Cancer. 2005; 114:190-4) (Raynaud J P, Cousse H, Martin P M. Inhibition of type 1 and type 2 5alpha-reductase activity by free fatty acids, active ingredients of Permixon. J Steroid Biochem Mol Biol. 2002; 82:233-9). It also contains phytosterols with anti-inflammatory action, such as beta-sitosterol, which have demonstrated beneficial effects in the treatment of Benign Prostatic Hiperplacia (Berges R R, Kassen A, Senge T. Treatment of symptomatic benign prostatic hyperplasia with beta-sitosterol: an 18-month follow-up. BJU Int. 2000; 85:842-6). It also contains beta-carotene, which modulates the growth of human prostate cancer cells (Williams A W, Boileau T W, Zhou J R. Beta-carotene modulates human prostate cancer cell growth and may undergo intracellular metabolism to retinol. J Nutr. 2000; 130:728-32). A Meta-Analysis carried out by the Department of Veterans Affairs Coordinating Center of the Cochrane Collaborative Review Group in Prostatic Diseases and Urologic Malignancies, Minneapolis Veterans Affairs Medical Center, USA—analyzed a total of 18 randomized controlled trials involving 2939 men and reported:

“As compared with men receiving placebo, men treated with S repens had decreased urinary tract symptom scores, Nycturia, improvement of urinary tract symptoms and peak urine flow. The evidence suggests that S. repens improves urologic symptoms and flow measures” (Wilt T J, Ishani A, Stark G. Saw palmetto extracts for treatment of benign prostatic hyperplasia: a systematic review. JAMA. 1998; 280:1604-9). Serenoa contains at least 13 active principles.

Silybum marianum (Carduus marianus, Holy thistle, Marian thistle, and Milk thistle) The main active principles are: flavonolignans, including Silibine, Silibinin, Silicristine, Isosilibinin and Silidianin, collectively known as Sylimarin. Mechanisms which explain its therapeutic properties are diverse and include: anti-oxidation; lipidic anti-peroxidation. Anti-inflammatory effects are due to mastocytes stabilization, inhibition of neutrophils, and strong inhibition of leucotrien and prostaglandin formation. These actions have demonstrated this plants utility in prostate cancer prevention and treatment (Davis-Searles P R, Nakanishi Y, Kim N C. Milk thistle and prostate cancer: differential effects of pure flavonolignans from Silybum marianum on antiproliferative end points in human prostate carcinoma cells. Cancer Res. 2005; 65:4448-57). Extensive studies with Prostate cancer have also shown that inhibition of mitogenic and cell survival signaling are the most likely molecular targets of silibinin's efficacy in PCA. Silibinin inhibits prostate tumor growth without any apparent signs of toxicity (Singh R P, Agarwal R. A cancer chemopreventive agent silibinin, targets mitogenic and survival signaling in prostate cancer. Mutat Res. 2004; 555:21-32). The down-regulation of PSA by silibinin and its counteraction on DHT effects indicate that this compound can interact with the expression of genes that are regulated through the androgen receptor. Silibinin can also inhibit the telomerase activity that mediates cell immortality and carcinogenesis (Thelen P, Wuttke W, Jarry H. Inhibition of telomerase activity and secretion of prostate specific antigen by silibinin in prostate cancer cells. J Urol. 2004; 171:1934-8). Silibinin/silymarin also inhibits the secretion of proangiogenic factors from tumor cells, and causes growth inhibition and apoptotic death of endothelial cells accompanied by disruption of capillary tube formation.

More importantly, silibinin inhibits the growth of in vivo advanced human prostate tumor (Singh R P, Agarwal R. Prostate cancer prevention by silibinin. Curr Cancer Drug Targets. 2004; 4:1-11). One study assessed and compared the anticancer efficacy and associated mechanisms of silymarin and silibinin in human prostate cancer (PCA) PC3 cells. Silymarin and silibinin inhibited cell proliferation, induced cell death, and caused G1 and G2-M cell cycle arrest in a dose/time-dependent manner. These findings indicate that silymarin and silibinin modulate G1 phase cyclins-CDKs-CDKIs for G1 arrest, and the Chk2-Cdc25C-Cdc2/cyclin B1 pathway for G2-M arrest, together with an altered subcellular localization of critical cell cycle regulators (Deep G, Singh R P, Agarwal C. Silymarin and silibinin cause G1 and G2-M cell cycle arrest via distinct circuitries in human prostate cancer PC3 cells: a comparison of flavanone silibinin with flavanolignan mixture silymarin. Oncogene. 2006; 25:1053-69). Silybum provides at least 57 active principles in a single therapeutic.

Urtica dioica L. (European Nettle, Stinging Nettle) Its main active principles are insaturated fatty acids (linoleic-acid, linolenic-acid, oleic-acid) and saturated fatty acids (palmitic-acid). These acids have demonstrated inhibiting action over 5-Alpha-Reductase, enzyme which converts Testosterone into Dihydrotestosterone, the main hormone related with prostatic hiperplasia (Liang T, Liao S. Inhibition of steroid 5 alpha-reductase by specific aliphatic unsaturated fatty acids. Biochem J. 1992; 285:557-62). It also contains phytosterols with anti-inflammatory action such as beta-sitosterol, beneficial in the treatment of benign prostatic hyperplasia (Berges R R, Kassen A, Senge T. Treatment of symptomatic benign prostatic hyperplasia with beta-sitosterol: an 18-month follow-up. BJU Int. 2000; 85:842-6). It also contains lycopene. Epidemiological studies have shown an inverse association between dietary intake of lycopene and prostate cancer risk. A clinical, randomized, controlled trial reports that Lycopene may have beneficial effects in prostate cancer (Kucuk O, Sarkar F H, Djuric Z. Effects of lycopene supplementation in patients with localized prostate cancer. Exp Biol Med (Maywood). 2002; 227:881-5). Contains 73 active principles.

Vitamin E is a micronutrient antioxidant that protects cells from oxidative damage involved in prostate carcinogenesis. A substantial amount of epidemiologic, molecular, and clinical evidence studies have repeatedly associated a high intake of vitamin E with reduced prostate cancer risk (Klein E A. Can prostate cancer be prevented? Nat Clin Pract Urol. 2005; 2:24-31) (Kirsh V A, Hayes R B, Mayne S T. Supplemental and dietary vitamin E, beta-carotene, and vitamin C intakes and prostate cancer risk. J Natl Cancer Inst. 2006; 98:245-54) (Wolk A. Diet, lifestyle and risk of prostate cancer. Acta Oncol. 2005; 44:277-81). Vitamin E significantly inhibits proliferation and induces apoptosis of prostate cancer cell lines in a dose and time dependent manner. Induced apoptosis is mediated by inhibition of Bcl-xL/Bcl-2 function leading to caspase-4 dependent apoptosis in prostate tumor cells. Vitamin E significantly suppressed tumor growth as well as lung metastases. (Malafa M P, Fokum F D, Andoh J. Vitamin E succinate suppresses prostate tumor growth by inducing apoptosis. Int J Cancer. 2006; 118:2441-7) (Shiau C W, Huang J W, Wang D S. alpha-Tocopheryl succinate induces apoptosis in prostate cancer cells in part through inhibition of Bcl-xL/Bcl-2 function. J Biol Chem. 2006; 281:11819-25).

Zinc is an essential metal for all cells. It plays a role in a wide variety of physiological and biochemical processes. In the prostate epithelial cell the accumulation of high cellular zinc is a specialized function that is necessary for these cells to carry out the major physiological functions of production and secretion of citrate. The production of citrate and its secretion into prostatic fluid is a differentiated function of the prostate epithelial cells that is important for reproduction. The normal human prostate accumulates the highest levels of zinc of any soft tissue in the body. It has been demonstrated that prostate malignant epithelial cells contain much less cellular zinc than the surrounding normal epithelial cells (Huang L, Kirschke C P, Zhang Y. Decreased intracellular zinc in human tumorigenic prostate epithelial cells: a possible role in prostate cancer progression. Cancer Cell Int. 2006; 6:10). The loss of citrate and zinc accumulation is the most consistent and persistent characteristic of prostate malignancy. This characteristic of prostate cancer indicates that the lost ability of the malignant cells to accumulate zinc and citrate is an important factor in the development and progression of malignancy. The lost ability of the epithelial cells to accumulate zinc and thus to also accumulate citrate is the result of decreased expression of specific zinc uptake transporters (Franklin R B, Feng P, Milon B. hZIP1 zinc uptake transporter down regulation and zinc depletion in prostate cancer. Mol Cancer. 2005; 4:32) (Costello L C, Franklin R B, Feng P. Cancer Causes Control. 2005; 16:901-15).

### EXAMPLE 2

### Composition—Prostate Disorders

A particularly preferred composition is shown in Table 1. Ratios reflect the concentration of active ingredient over the natural state, and the amounts provided are mg of extract. Obviously, the amount should be increased where the strength is reduced, and vice versa.

A particularly preferred composition is shown in Table 1.

TABLE 1
Composition

| Active Agent | Ratio | Amount (mg) |
|---|---|---|
| Energy enhancers | | |
| Panax ginseng | 5:1 | 57 |
| Pfaffia paniculata | 4:1 | 2 |
| Rhaponticum carthamoides | 6:1 | 2 |
| Bio-Intelligence modulators | | |
| Ganoderma lucidum | 6:1 | 143 |
| Grifola frondosa | 5:1 | 57 |
| Vitex agnus castus | 5:1 | 2 |
| Organization improvers | | |
| Arcostaphylos uva ursi | 4:1 | 71 |
| Curcubita pepo | 5:1 | 57 |
| Pygeum africanum | 5:1 | 172 |
| Selenium | 1:1 | 0.2 |
| Serenoa repens | 5:1 | 172 |
| Silybum marianum | 5:1 | 57 |
| Urtica dioica | 4:1 | 71 |
| Vitamin E | 1:1 | 28 |
| Zinc | 1:1 | 8.8 |
| Total | | 900 |

### EXAMPLE 3

### Prostate Disorders

Effectiveness and Tolerability Study

The effectiveness of the erectile dysfunction synergistic composition, formulated under the Systemic Medicine principles, was evaluated thought a retrospective, multicenter, descriptive two year study of 72 patients with erectile dysfunction. The composition improved erectile dysfunction in 90.3% of the patients; also, there was a marked improvement in Quality of Life. No side or secondary effects were observed in 98.6% of the study group. However, the significance of the observed effects in the other 7 patients did not warrant the suspension of the treatment. The erectile dysfunction formulation demonstrated to be an extraordinary treatment for patients with erectile dysfunction, reason why it should be considered as a treatment.

### EXAMPLE 4

### Principles for Selecting Synergistic Combinations

To explain the formulation encompassed by the invention, beneficial plants and nutraceuticals have been categorized into one of three groups, each of which should be present for synergistic effect. The classifications are: Energy, Bio-Intelligence and Organization. Plants and nutraceuticals classified under Energy are associated with ATP synthesis (such as the Krebs cycle, oxidative phosphorylation, beta-oxidation, etc.). Plants and nutraceuticals classified under Bio-Intelligence are those that regulate the neuroendocrine and immunological systems and cellular processes, thus controlling the interactions between the various systems in the body. Finally, plants and nutraceuticals classified under Organization are those that relate to the structure and function of specific organs. Combinations of plants and nutraceuticals from these three classification groups have synergistic effect because they address each necessary component of cellular and organic health—in effect they provide the triangle on which healing is fully supported. To illustrate this, the figure is provided below:

An illustrative example of synergy in medicinal plants is an in vitro study that demonstrates how the activity of herbal Berberine alkaloids is strongly potentiated by the action of 5′-methoxyhydnocarpin (5′-MHC)—an active principle of another phytomedicine (denominated Hydnocarpus wightiana). It shows a strong increase of accumulation of berberine in the cells in the presence of 5′-MHC, indicating that this plant compound effectively disabled the bacterial resistance mechanism against the berberine antimicrobial, thus showing the synergy of both substances. Stermitz F R, et al., Synergy in a medicinal plant: antimicrobial action of berberine potentiated by 5′-methoxyhydnocarpin, a multidrug pump inhibitor. Proc Natl Acad Sci USA. 2000; 97:1433-7.

A further demonstration may be provided of synergistic effect on a molecular scale by studying the gene expression profile changes in response to various plant ingredients and combinations thereof. Experiments are already underway demonstrating the expression profile in response to the formulations.

I will be aided in this work because researchers have already begun studying the expression profiles of various medicinal plants, thus providing a database of knowledge from which to build. E.g., Gohil, et al., mRNA Expression Profile of a Human Cancer Cell Line in Response to Ginkgo Biloba Extract: Induction of Antioxidant Response and the Golgi System, Free Radic Res. 2001; 33:831-849.

Finally there may be further presentation of gene expression results using whole-genome microarray analysis to demonstrate the formulation's capability to provide gene activation (upregulation or downregulation).

What is claimed is:

1. A phyto-nutraceutical composition, comprising plants or extracts or active ingredients derived from each of the following plants and nutraceuticals: Panax, Rhapontium, Ganoderma, Grifola, Vitex agnus castus, Arcostaphylos uva ursi, Curcubita, Pygeum, Selenium, Serenoa, Silybum, Urtica, Vitamin E and Zinc together with pharmaceutically acceptable excipients.

2. The phyto-nutraceutical composition of claim 1, further comprising: Panax ginseng, Panax quinquefolius, Rhapontium carthamoides, Ganoderma lucidum, Grifola frondosa, Vitex agnus castus, Arcostaphylos uva ursi, Curcubita pepo, Pygeum africanum, Selenium, Serenoa repens, Silybum marianum, Urtica dioica, Vitamin E and Zinc together with pharmaceutically acceptable excipients.

3. The phytoceutical composition of claim 2, comprising the relative amounts of ingredients shown in Table 1, and optionally including water or gelatin.

4. A method of treating disease comprising administering an effective amount of the composition of claim 3 to a patient sufficient to alleviate said disease.

5. The method of claim 4, wherein the diseases are prostate disorders.

6. A phyto-nutraceutical synergistic composition elaborated according to a method—Systemic Medicine—for the treatment of prostate disorders comprising: Panax ginseng, Panax quinquefolius, Rhaponticum carthamoides, Ganoderma lucidum, Grifola frondosa, Vitex agnus castus, Arcostaphylos uva ursi, Curcubita pepo, Pygeum africanum, Selenium, Serenoa repens, Silybum marianum, Urtica dioica, Vitamin E and Zinc together with pharmaceutically acceptable excipients.

7. The composition included in claim 6 wherein said composition preferably contains: Panax ginseng 57 mg, Pfaffia paniculata 2 mg, Rhaponticum carthamoides 2 mg, Ganoderma lucidum 143 mg, Grifola frondosa 57 mg, Vitex agnus castus 2 mg, Arcostaphylos uva ursi 71 mg, Curcubita pepo 57 mg, Pygeum africanum 172 mg, Selenium 0.2 mg, Serenoa repens 172 mg, Silybum marianum 57 mg, Urtica dioica 71 mg, Vitamin E 28 mg and Zinc 8.8 mg; together with pharmaceutically acceptable excipients.
